(19) United States
(12) Patent Application Publication
(10) Pub. No.: US 2025/0235420 A1
(43) Pub. Date: Jul. 24, 2025

(54) Phosphate and Arginine Containing Compositions and the Delivery of Such Compositions for Virulence Suppression

(71) Applicant: Solventum Intellectual Properties Company, Maplewood, MN (US)

(72) Inventors: Ranjani V. Parthasarathy, Woodbury, MN (US); Hannah C. Cohen, St. Paul, MN (US); Max A. Kruziki, New Brighton, MN (US)

(21) Appl. No.: 18/702,183

(22) PCT Filed: Oct. 20, 2021
(86) PCT No.: PCT/US2021/071937

§ 371 Date: Apr. 17, 2024

(51) Int. Cl. A61K 31/198; A61K 9/06; A61K 33/42; A61K 47/38; A61P 31/04

(52) U.S. Cl. CPC A61K 31/198; A61K 9/06; A61K 33/42; A61K 47/38; A61P 31/04

(57) ABSTRACT

Phosphate and arginine-containing compositions and methods of suppressing microbial virulence are provided. By suppressing virulence, administration and/or application of the medical compositions can be used to prevent, mitigate, or treat a microbial infection.

### BACKGROUND

Many known treatments of pathogens result in the destruction of all or nearly all microbes that may be present, even beneficial microbes. Further, because of these treatment methods, there is growing concern about antibiotic resistance that may increase risks to patients, particularly to those undergoing surgical procedures. Newer approaches have been directed toward suppressing the virulence of the pathogen that causes the infection rather than destroying all microbes.

New methods are needed to prevent the expression of one or more virulence factors while preserving colonization of beneficial bacteria. That is, new methods are needed that do not destroy all the beneficial bacteria in the process of preventing the harm done by pathogens. Phosphate-containing compositions for virulence suppression are described in U.S. Patent Application Publication No. 2019/0247423 to Alverdy et al.

### SUMMARY

Phosphate and arginine-containing compositions and methods of suppressing microbial virulence are provided. The medical compositions can be administered and/or applied in various formulations such as a gel (e.g., cellulosic gel), a spray, lotion, ointment, solution, emulsion, dispersion, foam, coating, paste, powder, tablet, capsule, or the like. The formulation used can be chosen based upon the location of the infection or potential infection and on the desired delivery method.

By suppressing virulence, administration and/or application of the medical compositions can be used to prevent, mitigate, or treat a microbial infection. More specifically, the medical compositions include phosphate and arginine. The phosphate and arginine-containing compositions can suppress the expression of various virulence factors without destroying all microbes that may be present.

Phosphate and arginine supplementation can reduce, inhibit, or prevent the expression of Pseudomonas aeruginosa pyoverdine and Pseudomonas aeruginosa and Enterococcus faecalis collagenase activity. Pyoverdine is a fluorescent siderophore produced by Pseudomonas aeruginosa that is involved in iron scavenging. Breakdown of tissue proteins has been observed in many bacterial infections and is attributed to bacterial collagenase activity.

In embodiments, phosphate and arginine are available in combination on a tissue for a period of days to suppress virulence. When phosphate and arginine are used separately, they can get redistributed in the gut or washed away at the wound site. The delivery of phosphate and arginine to suppress virulence slowly and consistently can reduce, inhibit, or prevent microbial virulence in humans. While one skilled in the art would anticipate that phosphate and arginine form a stable bond, which can have “covalent like” stability when used together at neutral pH, and thereby would be unavailable as a nutrient to bacteria for preventing virulence, it was surprising to see that a phosphate-arginine composition with certain ratios as described herein resulted in good virulence suppression for both Pseudomonas aeruginosa pyoverdine and collagenase assays. Furthermore, a cellulosic gel was used as a vehicle for delivery of arginine and phosphate where there was good performance over a long period.

The terms “Pi” or “pi” used herein refers to phosphate.

The terms “Arg” or “arg” used herein refers to L-arginine.

The term “mM” used herein refers to millimolar concentration.

The term “virulence” refers to a pathogen's ability to infect or damage a host such as a mammal. The term “virulence suppression” and “suppression of microbial virulence” or similar expressions refer to suppressing or inhibiting the synthesis and/or expression of one or more virulence factors.

The term “virulence factor” refers to molecules produced by microbes that enable them to infect a host such as a mammal. The virulence factors of bacteria can be small molecules, proteins, or biofilms (e.g., a slimy buildup of bacteria on a surface). The virulence factors are typically secreted by a microbe to promote colonization and/or adhesion to a host (e.g., resulting in biofilm formation), to evade the immune response of the host, or to obtain nutrients from the host.

The terms “comprise”, “contain”, “include”, and variations thereof do not have a limiting meaning where these terms appear in the description and claims. Such terms will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and is limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether they materially affect the activity or action of the listed elements. Any one of the elements or combinations of elements that are recited in this specification in open-ended language (e.g., comprise, include, contain, and derivatives thereof), are considered to additionally be recited in closed-ended language (e.g., consist and derivatives thereof) and in partially closed-ended language (e.g., consist essentially, and derivatives thereof).

The words “preferred” and “preferably” refer to embodiments of the disclosure that may afford certain benefits, under certain circumstances. However, other claims may also be preferred, under the same or other circumstances. Furthermore, the recitation of one or more preferred claims does not imply that other claims are not useful and is not intended to exclude other claims from the scope of the disclosure.

In this application, terms such as “a”, “an”, and “the” are not intended to refer to only a singular entity but include the general class of which a specific example may be used for illustration. The terms “a,” “an,” and “the” are used interchangeably with the term “at least one.” The phrases “at least one of” and “comprises at least one of” followed by a list refers to any one of the items in the list and any combination of two or more items in the list.

As used herein, the term “or” is generally employed in its usual sense including “and/or” unless the content clearly dictates otherwise. The term “and/or” means one or both. For example, the expression A and/or B means A alone, B alone, or both A and B.

Also, the recitations of numerical ranges by endpoints include all numbers subsumed within that range as well as the endpoints (e.g., 1 to 5 includes 1, 1.5, 2, 2.75, 3, 3.80, 4, 5, etc.) and any sub-ranges (e.g., 1 to 5 includes 1 to 4, 1 to 3, 2 to 4, etc.).

### DETAILED DESCRIPTION

The importance of the amount of available phosphate in the proximity of bacteria has recently been demonstrated by various researchers. The virulence activity of certain bacteria such as Pseudomonas aeruginosa can increase if phosphate in their environment is scarce but can decrease if phosphate is abundant. For example, if a mammalian gut experiences a physiologic stress such as surgery, phosphate can become depleted. This depletion triggers colonized bacteria to express certain virulence factors. Hence, medical compositions that can provide a phosphate supply to the gut are critically needed. One approach to providing the phosphate supply is to administer a medical composition that can coat the gut and prevent the bacteria from turning virulent. Similarly, medical compositions that can supplement phosphate in a wound environment or surgical site would also be useful in keeping bacteria such as Pseudomonas aeruginosa from turning virulent.

Arginine is a key amino acid and a precursor to nitric oxide, which increases blood flow and oxygen to the wound. Arginine can increase collagen formation and reduce inflammation and can improve healing in wounds.

Phosphate and arginine-containing compositions and methods of suppressing microbial virulence are provided. Microbial virulence is suppressed by reducing or inhibiting the formation and/or expression of one or more virulence factors, which are the harmful products that can lead to microbial infections. That is, the medical compositions can prevent, mitigate, or treat microbial infections. The medical compositions typically do not prevent continued colonization of microbes such as those that are helpful to the mammal.

### Medical Compositions

In another aspect, a medical composition is provided that includes any one of the phosphate and arginine compositions that are described above. L-arginine is present as positively charged cation and is associated with a negatively charged counter-ion, i.e. a phosphate anion. The phosphate anion can neutralize the basic centers of arginine. One or a plurality of different phosphate and arginine-containing compositions can be used in the medical compositions. Two or more different phosphate and arginine-containing compositions can be blended within the medical composition to suppress virulence of different types of pathogens and/or to suppress different virulence factors expressed by a single type of pathogen. For example, a first phosphate or arginine-containing composition that is particularly effective at suppressing a first virulence factor can be combined with a second phosphate or arginine-containing composition that is particularly effective at suppressing a second virulence factor. The plurality of different phosphate-containing compositions can be blended in any desired ratio. Arginine phosphate compositions can be prepared by adding arginine in optimal ratios as described herein with phosphoric acid/phosphate buffer that can then release arginine and phosphate to suppress virulence factors. In some cases, additional salts such as chloride may be present in solution. It is also possible to use salts L-arginine forms with pharmaceutically acceptable organic acids such as acetic acids, ascorbic acid, lactic acid, tartaric acid, malic acid, oxalic acid or glutamic acid with arginine phosphate compositions. The medical composition can contain 0.05 to 100 weight percent of arginine phosphate salt based on a total weight of the medical composition. The amount can be, at least about 0.05 weight percent, at least about 0.1 weight percent, at least about 0.5 weight percent, at least about 1 weight percent, at least about 2 weight percent, at least about 5 weight percent, at least about 10 weight percent, at least about 20 weight percent, at least about 30 weight percent, at least about 40 weight percent, or at least about 50 weight percent and up to 100 weight percent, up to 90 weight percent, up to 80 weight percent, up to 70 weight percent, up to 60 weight percent, or up to 50 weight percent.

The phosphate and/or arginine-containing composition can be purified, if desired, using any method that is suitable for purification of polymeric materials included in a medical composition. For example, the phosphate and/or arginine-containing composition can be purified by filtration.

The medical composition can optionally further include other components that facilitate delivery of the medical composition for preventing, mitigating, or treating a microbial infection. The optional components are selected to be therapeutically acceptable, which means that the optional components do not interfere with the effectiveness of the phosphate and/or arginine-containing composition and are not toxic to the mammal being treated. The additional components are typically selected so that the medical composition does not substantially reduce non-pathogenic and/or normally helpful microbes that may be present. The log reduction of microbes is often less than 1.

The medical compositions can be administered and/or applied in any desired formulation such as a spray, lotion, ointment, gel, solution, emulsion, dispersion, foam, coating, paste, powder, tablet, capsule, or the like. The formulation used is dependent on the location of the infection or potential infection and on the desired delivery method.

For some applications, it is desirable that the medical composition remain in a location where is it administered and/or applied. Such medical compositions are usually formulated to have a suitably high viscosity and/or to include a hydrophobic component that will enhance retention of the medical composition at the application location. These formulations can be, for example, an emulsion, ointment, gel, or lotion. Emulsions can be oil-in-water or water-in-oil.

The medical compositions that include components such as, for example, water, organic solvents, hydrophobic components (e.g., petrolatum and oils,), hydrophilic components (glycerin and various ether and/or polyether compounds), surfactants (i.e., anionic, cationic, non-ionic, amphoteric, and ampholytic surfactants), silicones, carbohydrates (polysaccharides such as hydroxypropyl methyl cellulose), thickeners such as carbopol, film-formers, emulsifiers, water, organic solvents (e.g., alcohols and polyols), stabilizers (e.g., polymers), fillers (e.g., organic materials such as polymeric particles and inorganic materials including ceramic particles, silica particles, clay particles, and glass particles), emollients/moisturizers, humectants, tonicity adjusting agents, chelating agents, anti-inflammatory agents, gelling agents, preservatives, pH adjusting agents, viscosity builders, time-release agents, dyes, fragrances or oils, and the like.

The medical compositions optionally can be sterilized by any suitable method that will not negatively impact its efficacy. For example, if desired, the medical composition can be treated with ethylene oxide.

Method of Administering and/or Applying the Medical Composition

In another aspect, a method of suppressing microbial virulence is provided. The microbial virulence is typically suppressed by reducing or inhibiting the synthesis and/or expression of one or more virulence factors by the microbe. By suppressing the synthesis and/or expression of one or more virulence factors, a microbial infection can be prevented, mitigated, or treated.

The method includes administrating and/or applying a medical composition comprising a phosphate and arginine-containing composition. In embodiments, the composition comprises between about 5 mM to about 150 mM arginine and about 5 mM to about 150 mM phosphate. In further embodiments, the composition comprises between about 25 mM to about 150 mM arginine and about 10 mM to about 150 mM phosphate. In yet further embodiments, the composition comprises between about 50 mM to about 150 mM arginine and about 50 mM to about 150 mM phosphate. In embodiments, the composition comprises between about 90 mM to about 100 mM arginine and about 130 mM to about 140 mM phosphate. In an embodiment, the composition comprises about 100 mM arginine and about 100 mM phosphate. In another embodiment, the composition comprises about the same amount (mM) of arginine and phosphate. In yet another embodiment, the composition comprises about the same amount (mM) or higher of arginine relative to phosphate.

In some embodiments, the medical compositions may optionally include a thickener (at least 0.01 wt. %, at least 0.05 wt. %, at least 0.1 wt. %, or at least 1 wt. %, based on the total weight of the composition). In such embodiments, toxicity considerations may be mitigated due to release profile, allowing for increased levels of arginine or phosphate in the composition. In such embodiments, the composition may include between about 5 mM to about 700 mM arginine and about 5 mM to about 700 mM phosphate.

Thickeners, such as hydroxypropyl methylcellulose, can be added to the arginine phosphate compositions to increase the viscosity of the composition. In general, the polymers useful as thickeners have sufficient molecular weight to achieve thickening at generally less than 5 wt-% polymer, but not too high that the composition feels slimy and stringy. While the composition of the polymer will dramatically affect the molecular weight at which sufficient thickening will occur, the polymers preferably have a molecular weight of at least 250,000 daltons, and more preferably at least 500,000 daltons. The polymers preferably have a molecular weight of no greater than 3,000,000 daltons, and more preferably no greater than 1,000,000 daltons.

Polymers used to thicken solutions can be classified as soluble, swellable, or associative in the aqueous compositions. Some polymers may fall into one or more of these classes. For example, certain associative polymers can be soluble in the aqueous system. Whether they are considered soluble, swellable, or associative in the aqueous system, suitable polymers may be film forming or not. Film forming polymers may retain the active virulence suppression component at the afflicted site for longer periods of time. This may be desirable for certain applications. For example, some film forming polymers may produce compositions that could not be easily washed off with water after being applied and dried.

As used herein, a soluble polymer is one that in dilute solution (i.e., 0.01-0.1 wt-% in the desired aqueous solvent system defined as containing water and any other hydrophilic compounds), after heating for a sufficient time to ensure solubilization of any potentially soluble components, has no significant observable particles of greater than 1 micron in particle size, as determined by light scattering measurements using, for example, Malvern Masterisizer E Laser Particle Size Analyzer available from Malvern Co., Boston, Mass.

As used herein, a swellable polymer is one that in dilute solution (i.e., 0.01-0.1 wt-% in the desired aqueous solvent system), after heating for a sufficient time to ensure solubilization of any potentially soluble components, has a significant (i.e., detectable) number of observable particles of greater than 1 micron in particle size, as determined by light scattering measurements using, for example, Malvern Masterisizer E Laser Particle Size Analyzer.

Thickeners can be nonionic polymeric thickeners. Thickeners that are salt tolerant can be used.

A group of nonionic polymeric thickeners include modified celluloses, guar, xanthan gum, and other natural polymers such as polysaccharides and proteins, associative polymers based on nonionic ethylenically unsaturated monomers wherein at least one comonomer has at least 16 carbon atoms, and polymers based on ethylenically unsaturated monomers selected from the group consisting of acrylates, acrylamides, vinyl lactams, vinyl acetate and its hydrolyzed derivatives, methyl vinyl ethers, styrene, and acrylonitrile.

Examples of nonionic soluble polymers include a variety of cellulosic ethers are reported in the literature to be soluble in water. Materials in this class that are nonionic and have been shown to be useful include: Hydroxypropyl methyl cellulose available as METHOCEL K100M Premium from Dow Chemical Company, Midland, MI, methylhydroxypropylcellulose, available as BENECEL MP 943 from Aqualon, Wilmington, Del.; hydroxypropylcellulose, available as KLUCEL (LF, GF, MF, HF) from Aqualon; hydroxybutylmethylcellulose (3.5 wt-% hydroxybutyl and 30 wt-% methoxyl) from Scientific Polymer Products, Ontario, N.Y.; and hydroxyethylcelluloses, available under the trade designation NATROSOL from Aqualon. Xanthan gum, guar, locust bean gum, and other polysaccharides may also be suitable. These polymers may be produced from plant sources or can be produced through microbial cell culture. Polyvinyl alcohol (PVA) also may be suitable. For example, PVA made from polyvinyl acetate, which has been hydrolyzed to about 87%, is highly water soluble at room temperature. Those with higher percent hydrolysis become progressively more crystalline and may need to be heated to get into solution. Protein thickeners such as gelatin and pectin may also be useful. Other soluble polymers include amine oxide polymers such as those described in U.S. Pat. No. 6,123,933 and those commercially available under the trade designation DIAFORMER Z-711, Z-712, Z-731, and Z-751 from Clariant Corp. are useful. Additionally, zwitterionic polymers, such as methacryloyl ethyl betaine/acrylate copolymer that are commercially available under the trade designation DIAFORMER Z-400 from Clariant Corp. can also be used. Zwitterionic polymers described in U.S. Pat. No. 6,590,051 may also be useful.

Chemical crosslinking using polyunsaturated monomers such as diallyl maleate may also prove useful. Other suitable crosslinkers are multi-ethylenically unsaturated compounds wherein the ethylenic groups are vinyl groups (including substituted vinyl groups, such as isopropenyl groups), allyl groups, and/or methallyl groups, which groups are bonded to nitrogen or oxygen atoms. Vinyl, allyl, and methallyl groups, as used herein, include substituted derivatives. Exemplary compounds include divinyl, diallyl, or dimethallyl esters, ethers, amides, or ureas. Specific examples are disclosed in U.S. Pat. No. 5,225,473 (Duan) and U.S. Pat. No. 4,931,282 (Asmus et al.). A range of crosslinked polyvinylpyrrolidone (PVP) materials has been prepared via covalent crosslinking with diallyl maleate or by radiation crosslinking of linear PVP powders. Crosslinked PVP prepared under these techniques can produce colloidal particles which are highly swellable in aqueous solutions and thereby produce viscous solutions. The polymers are also nonionic and have excellent compatibility with cationic excipients.

Associative polymers can be used to thicken the compositions of the present disclosure as well. Such polymers thicken as a result of hydrophobic or van der Waals association of hydrophobic side chains. Such associative polymers can form viscous to gelled aqueous solutions despite their relatively low molecular weights. A class of such associative polymers is based on nonionic ethylenically unsaturated monomers wherein at least one comonomer has at least 12 and preferably at least 16 carbon atoms. An example is cetyl hydroxyethylcellulose, available as NATROSOL PLUS from Aqualon, which utilizes an associative mechanism to enhance the viscosity it produces.

Bioadhesive polymers optionally may be added to the neat compositions as well as the other physical forms. Numerous suitable bioadhesive polymers are discussed in WO 93/21906.

Carboxylic acid functional polymers including naturally occurring carboxylic acid functional polymers such as hyaluronic acid and derivatives of natural polymers such as carboxymethylcellulose, alginic acid and other alginate polymers, Fucogel (a polysaccharide consisting of three mono-saccharides, fucose, galactose, and galacturonic acid), hyaluronic acid, and the like, also may be useful. Synthetic polymers may also be useful, such as those based on carboxylic acid, phosphonic acid, or sulfonic acid functional monomers, including but not limited to, polymers derived from acrylic acid, methacrylic acid, maleic anhydride, itaconic anhydride, sodium AMPS (the sodium salt of 2-acrylamido-2-methylpropane sulfonic acid), sulfopropyl acrylate or methacrylate, sulphomethylated acrylamide, allyl sulphonate, sodium vinyl sulphonate, combinations thereof, or other water-soluble forms of these or other polymerizable carboxylic or sulphonic acids. Anionic swellable polymeric thickeners may also be useful. Anionic polymers for virulence suppression include polymers having carboxylic acid, carboxylate, sulfonic acid, sulfonate, phosphonic acid or phosphate groups.

A group of cationic polymeric thickeners include cationically modified celluloses, quaternized natural amino-functional polymers, and polymers based on ethylenically unsaturated monomers selected from the group consisting of acrylates, acrylamides, vinyl lactams, vinyl acetates, methyl vinyl ethers, styrene, and acrylonitrile. Cationic polymers for use in the compositions of this disclosure can be selected from both permanently charged quaternary polymers (those polymers with quaternary amines such as Polyquaternium 4, 10, 24, 32, and 37, described below) as well as protonated primary, secondary, and tertiary amine functional polymers that have been protonated with a suitable protonic acid. The quaternary, tertiary, secondary, and primary amine functional polymers may be chosen from natural polymers, modified natural polymers, as well as synthetic polymers. Examples of swellable polymers include modified cellulose products are sold under the trade names CELQUAT (National Starch and Chemicals Corp., Bridgewater, N.J.) and UCARE (Amerchol Corporation, Edison, N.J.), cationic polysaccharide polymer that can be used is a cationic guar gum derivative, such as guar hydroxypropyltrimonium chloride (commercially available from Rhone-Poulenc under the trade designation JAGUAR). A polymer is a poly(2-methacryloxyethyl trimethyl ammonium chloride) polydimethylaminoethyl methacrylate, which conforms to the CTFA designation Polyquaternium 37. Another polymer includes acrylamide and methacryloyloxyethyl trimethyl ammonium chloride, which conforms to the CTFA designation Polyquatemium 32. These are commercially available from Allied Colloids Inc. of Suffolk, Va. as SALCARE SC95, SC96, and SC92. Polymers of N-vinyl lactams, such as N-vinyl pyrrolidone, when exposed to gamma radiation increase in molecular weight and may actually crosslink. This crosslinking allows for more efficient thickening (less polymer required to achieve a certain viscosity) and an improved cosmetic feel. Other polymers that when exposed to gamma radiation result in crosslinking, include polymers such as LUVIQUAT HM 552 (copolymers of vinylimidazolium methochloride and vinylpyrrolidone, which conforms to the CTFA designation Polyquaternium-16), and GAFQUAT HS-100 (vinylpyrrolidone/methacrylamidopropyltrimethylammonium chloride copolymer which conforms to the CTFA designation Polyquaternium-28).

Any suitable method of administering and/or applying the medical composition can be used. For example, the medical composition can be applied to skin, mucosa, tissue (both exterior and interior surfaces of tissue), a wound site, a surgical site, an implant (e.g., knee and hip replacement, pacemaker, heart valve, or stent), a catheter, a suture, or a bone.

The medical compositions can be administered and/or applied locally or systemically. For example, the medical compositions can be applied using a swab, cloth, sponge, nonwoven wipe, paper product such as a tissue or paper towel, or the like. When applied locally, the medical composition desirably remains where it was applied. That is, the medical composition persists at the location for enough time to suppress virulence of the pathogen. In other examples, the medical composition can be administered orally or intravenously. For some infections, such as those that are initiated in the gut, the medical composition can be administered by drinking a solution or by swallowing a tablet or capsule.

For treatment of wounds and surgical sites, application of the medical composition as a coating may be desirable. Alternatively, the medical composition can be applied to a solid or porous support and then applied to the wound. Suitable supports include, for example, polymeric foams, polymeric films, and knitted or non-woven materials. The medical composition can be used for preventing and treating both acute and chronic wound infections and can be applied to any wound surface.

The medical composition can be administered and/or applied to reduce biofilm attachment on various surfaces. For example, the medical compositions can be applied to implants and catheters prior to their insertion into a mammalian body. In other examples, the medical compositions can be applied to bedding, surgical tables, tubing used in medical procedures, and other reusable medical equipment that contacts a mammal. In yet other examples, the medical compositions can be a liquid composition that is used to control or prevent biofilm populations in oral applications, such as for treating gingivitis. In still other examples, the medical compositions can be used to control or prevent biofilm populations in the middle ear that have been found in chronic otitis media. In yet other examples, the medical compositions can be used to control or prevent biofilm populations in the nose, which can result in the prevention or treatment of various infections such as those in the lungs and in blood. The medical compositions can often impact virulence factors either before or after biofilm formation.

The medical composition is suitable for preventing and treating urinary tract infections (e.g., administered in the form of a drink), ventilator associated pneumonia (e.g., administered in the form of a drink, tablet, or capsule), medical device/implant infections (e.g., administered by application as a coating on the medical device/implant), wounds (e.g., administered by application of a coating on the wound, whether chronic or acute), bloodstream infections (e.g., administered and/or applied to the blood-contacting tissue), mucosal tissue infections (e.g., administered in the nose), gastrointestinal tract (administered in the form of a coating, drink, tablet, or capsule), vaginal tissue (e.g., administered in the form of a coating), anastomotic tissue (e.g., administered as a coating on the surgical site to prevent anastomotic leaks), peritoneum (e.g., administered at the surgical site), sepsis, and the like. In some embodiments, where this is an existing microbial infection, the medical composition is applied over the area where the microbes are located.

The medical composition is usually administered in a therapeutically effective amount. This refers to the amount of the medical composition (or the amount of the phosphate and arginine-containing compositions) that is needed to inhibit the synthesis and/or expression of one or more virulence factors by a microbe or that is enough to reduce, mitigate, or prevent a microbial infection.

Administering the medical composition suppresses at least one type of virulence factor. That is, the medical composition suppresses the formation or expression of various molecules that may be harmful to the mammal and/or suppresses the formation of biofilms on a foreign object such as an implant suture in the mammal. For example, the medical composition can suppress the formation or expression of pyocyanin, pyoverdine, collagenase (which is often measured by breakdown of gelatin as a surrogate of collagenase activity), and biofilms by bacteria.

In many embodiments of administering the medical composition, the virulence factor is reduced by at least about 25 percent, 50 percent, at least about 60 percent, at least about 70 percent, at least about 75 percent, at least about 80 percent, at least about 90 percent, at least about 95 percent, at least about 99 percent, at least about 99.5 percent, or at least about 99.9 percent when compared to the vehicle only control. The percentage can be based on weight, area, volume, or any other suitable measurable amount such as the intensity of a fluorescent or absorbance signal indicating virulence activity.

The medical composition may be suitable for treating any known microbe including, for example, bacteria, viruses, fungi such as Candida, and mycobacteria. In particular, administrating the medical composition can suppress virulence of at least one of gram negative Pseudomonas aeruginosa, gram positive Enterococcus faecalis, gram positive Staphylococcus aureus, or gram negative Serratia marcescens

Unlike some previously known methods of treating microbial infections, the medical composition does not substantially kill all microbes within the treatment area. Although some of the pathogens may be destroyed at the treatment site such as those associated with a biofilm, colonization of the protective microbes is not substantially reduced. Stated differently, the pathogens can be contained and controlled while the colonization resistance of the non-pathogenic microbes and/or the normally protective microbes can be preserved. As used in reference to reduction in the number of microbes that are present, the term “substantially” means that there is less than 1 log reduction of the microbes. In some embodiments, there may be in increase in the growth of protective microbes.

### EXAMPLES

### Materials

| | | Vendor | Product |
|---|---|---|---|
| Material | Vendor | Location | Code |
| 2X Tryptone Yeast | Sigma Life Science | St. Louis, | Y1003- |
| Medium (2X TY) | | MO | 500ML |
| TSA (Trypticase Soy | Becton, Dickinson | Sparks, MD | 221283 |
| Agar) Plates | and Company | | |
| Tryptic Soy Broth (TSB) | General Laboratory | Yorkville, IL | 16T- |
| | Products | | 3800-5 |
| DQ gelatin from pig skin, | Invitrogen | Eugene, OR | D12054 |
| fluorescein conjugate | | | |
| Methocel K100M Premium | Dow Chemical | Midland, MI | |
| Hydroxypropyl | Company | | |
| methylcellulose (HPMC) | | | |
| L-arginine | Acros Organics | Fair Lawn, | |
| Enterococcus faecalis | | NJ | |
| V583 | | | |
| MPAO1-P2 Pseudomonas | | | |
| aeruginosa | | | |

### Preparation of Gel Extracts (Example 2)

Gels were prepared in a 50-mL conical tube and centrifuged briefly so they collected on the bottom of the tube. Sterile water was added to the top of the gel at a 1:2.5 ratio (e.g., 25 mL water for 10 mL gel) and the tubes were placed at 37° C. A 0.51 mL aliquot was collected from the top of the tube after gentle mixing at each extraction time point.

### Pyoverdine Assay (Examples 1 and 2)

An MPAO1-P2 Pseudomonas aeruginosa colony from a TSA plate was grown overnight in TY media (5 mL) with shaking at 37° C.

Individual growth media solutions for the assay were freshly prepared by adding potassium phosphate buffer and/or L-arginine to 10% TY media and then adjusting the pH of each solution to about pH 6.0 using acid or base. Gel extractions were prepared by diluting 20% TY media 1:1 with gel extractions and then adjusting the pH of each solution to about pH 6.0 using acid or base.

Each growth media solution (200 microliters) was added to a separate well of a 96-well black, clear-bottom plate. Samples were prepared in triplicate (n=3). MPAO1-P2 bacteria were centrifuged and resuspended in 5 mL of 10% TY media and 3 microliters of resuspended bacteria was added to each well. Background control wells were also prepared that did not have bacteria added to the wells. Pyoverdine production (fluorescent intensity at 360 nm excitation/460 nm emission) and bacteria growth (OD600; i.e., absorbance at 600 nm) were measured kinetically using a plate reader (Synergy HTX Plate Reader, Biotek Instruments, Winooski, VT) with shaking at 37° C. The background values were subtracted from the respective fluorescence and absorbance measurements. The pyoverdine fluorescence values (RFU, relative fluorescence units) were then normalized for bacteria growth by dividing the RFU value by the OD600 measurement. Data is shown at 21-24 hours.

### Collagenase Assay Using Gelatin Breakdown as a Surrogate (Examples 1 and 2)

Breakdown of tissue proteins has been observed in many bacterial infections and is attributed to bacterial collagenase activity. Gelatin breakdown was used as a surrogate for collagen breakdown in the assay. A fluorescently labeled gelatin (DQ gelatin-fluorescein conjugate) was used to monitor gelatin breakdown with the fluorescence signal increasing with increased gelatin degradation.

An MPAO1-P2 Pseudomonas aeruginosa colony was each obtained from a corresponding TSA plate and grown overnight in TY media (5 mL) with shaking at 37° C. Next, the culture was centrifuged at 3000×g for 5 minutes and the supernatant was removed. The bacteria were washed two times with water.

Individual growth media solutions for the assay were freshly prepared by adding potassium phosphate buffer and L-arginine to 10% TY media and then adjusting the pH of each solution to about pH 6.0 using acid or base. Gel extractions were prepared by diluting 20% TY media 1:1 with gel extractions and then adjusting the pH of each solution to about pH 6.0 using acid or base. All the growth media solutions were sterile filtered when possible using a 0.2 micrometer filter.

An aliquot of each growth media solution (200 microliters) was added to the well of a 96-well black, clear-bottom plate. Samples were prepared in triplicate (n=3). Reconstituted gelatin-fluorescein (20 microliters of 1 mg/mL) was then added to each well. The bacteria samples were resuspended in 5 mL of 10% TY media. A 3 microliter sample of resuspended MPAO1-P2 Pseudomonas aeruginosa was added to each well. Background control wells were also prepared that did not have bacteria added to the wells. Bacterial growth was measured at 600 nm (OD600) and collagenase activity was measured as fluorescent intensity at 485 nm excitation/528 nm emission. Collagenase activity (RFU) was normalized to bacterial growth (OD600) for each well. For MPAO1-P2 Pseudomonas aeruginosa, the time required to maximum fluorescence (i.e., time to inflection point or time to max slope) was measured.

### Example 1: Arginine Phosphate for Virulence Suppression

Arginine and phosphate buffer were mixed in the ratios defined and the pH was adjusted to 6-7. Compared to the control sample (10% TY media), the test samples with added arginine and phosphate did not substantially reduce P. aeruginosa bacterial growth (Tables 1, 2, 3). Varying the concentration of arginine and phosphate affects whether the cells produce pyoverdine and collagenase virulence factors (Tables 1, 2, 3). The data show that approximately equimolar arginine and phosphate provide optimum performance in both assays.

TABLE 1
MPAO1-P2 Growth (Background Subtracted) at 24 Hours (Column
2), Pyoverdine Production/Growth at 24 Hours (Column 3), and
Time to the Proteolytic Activity Inflection Point (Column 4)

| | Bacteria Growth | Pyoverdine | |
|---|---|---|---|
| | (Background | Production | Time to Proteolytic |
| | Subtracted OD600; | (RFU/OD600; | Activity Inflection |
| Material added to | 24 hours) | 24 hours) | Point (hours) |
| Growth Media | Mean ± Standard | Mean ± Standard | Mean ± Standard |
| (10% TY) | Deviation (n = 3) | Deviation (n = 3) | Deviation (n = 3) |
| None (control) 10 | 0.198 ± 0.011 | 39 ± 5 | 9.0 ± 0.1 |
| % TY | | | |
| 100 mM Arginine, | 0.397 ± 0.113 | 109 ± 35 | 17.3 ± 0.1 |
| 200 mM Pi | | | |
| 100 mM Arginine, | 0.17 ± 0.006 | 6 ± 6 | Did not reach inflection |
| 100 mM Pi | | | by 30.2 hours |
| 100 mM Arginine, | 0.18 ± 0.026 | 22 ± 9 | Did not reach inflection |
| 50 mM Pi | | | by 30.2 hours |
| 100 mM Arginine, | 0.183 ± 0.006 | 53 ± 7 | Did not reach inflection |
| 25 mM Pi | | | by 30.2 hours |

TABLE 2
MPAO1-P2 Growth (Background Subtracted) at 24 Hours (Column
2), Pyoverdine Production/Growth at 24 Hours (Column 3), and
Time to the Proteolytic Activity Inflection Point (Column 4)

| | Bacteria Growth | Pyoverdine | |
|---|---|---|---|
| | (Background | Production | Time to Proteolytic |
| | Subtracted OD600; | (RFU/OD600; | Activity Inflection |
| Material added to | 24 hours) | 24 hours) | Point (hours) |
| Growth Media | Mean ± Standard | Mean ± Standard | Mean ± Standard |
| (10% TY) | Deviation (n = 3) | Deviation (n = 3) | Deviation (n = 3) |
| None (control) 10 | 0.12 ± 0.02 | 346 ± 34 | 13 ± 3 |
| % TY | | | |
| 25 mM Arginine, | 0.295 ± 0.007 | 63 ± 3 | 29.7 ± 0.6 (Did not reach |
| 50 mM Pi | | | inflection by 30 hours in 2/3 |
| | | | replicates; averaged 29, 30, |
| | | | and 30) |
| 50 mM Arginine, | 0.231 ± 0.003 | 94 ± 6 | Did not reach inflection by 30 |
| 50 mM Pi | | | hours |
| 50 mM Arginine, | 0.332 ± 0.006 | 90 ± 5 | 20 ± 5 |
| 25 mM Pi | | | |

TABLE 3
MPAO1-P2 Growth (Background Subtracted) at 24 Hours (Column
2), Pyoverdine Production/Growth at 24 Hours (Column 3)

| | Bacteria Growth | Pyoverdine |
|---|---|---|
| | (Background | Production |
| | Subtracted OD600; | (RFU/OD600; |
| | 24 hours) | 24 hours) |
| Material added to Growth | Mean ± Standard | Mean ± Standard |
| Media (10% TY) | Deviation (n = 3) | Deviation (n = 3) |
| None (control) 10% TY | 0.16 ± 0.02 | 327 ± 36 |
| 5 mM Arginine, 5 mM Pi | 0.21 ± 0.01 | 209 ± 24 |
| 10 mM Arginine, 10 mM Pi | 0.27 ± 0.02 | 152 ± 18 |
| 25 mM Arginine, 25 mM Pi | 0.38 ± 0.02 | 83 ± 9 |

### Example 2: Thickened Composition of Arginine Phosphate for Virulence Suppression

Phosphoric acid was added to arginine solution and the pH was adjusted to 7 in 0.5%, 2%, and 4% HPMC. The solutions were autoclaved using a 121° C., 15-minute liquid cycle. Sterile water was added to the top of the 2% and 4% gels at a 1:2.5 ratio (e.g., 5 mL water for 2 mL gel) and the tubes were placed at 37° C. A 0.51 mL aliquot was collected from the top of the tube after gentle mixing at each extraction time point. These aliquots were diluted 1:1 in 20% TY media to evaluate pyoverdine production and collagenase activity in MPAO1-P2 P. aeruginosa. The 0.5% HPMC gels were directly diluted 1:1 in 20% TY media to evaluate pyoverdine production and collagenase activity in MPAO1-P2 P. aeruginosa. Solutions were prepared directly in 10% TY at the concentrations listed.

Compared to the control samples, the test samples with arginine phosphate did not substantially reduce bacterial growth (Table 4). Certain molar ratios of arginine/phosphate are more beneficial for virulence suppression and showed a dramatic reduction in pyoverdine production and an extended time to proteolytic activity (e.g., molar ratios of around 1:1 to 1:1.5 of Arg/Pi) (Table 4). Higher total concentrations of Arg/Pi are beneficial when the formulation is thickened due to slower release kinetics.

TABLE 4
MPAO1-P2 Growth (Background Subtracted) at 24 Hours (Column
2), Pyoverdine Production/Growth at 24 Hours (Column 3), and
Time to the Proteolytic Activity Inflection Point (Column 4)

| | Bacteria Growth | Pyoverdine | |
|---|---|---|---|
| | (Background | Production | Time to Proteolytic |
| Material/Gel Extract | Subtracted OD600; | (RFU/OD600; | Activity Inflection |
| Collected at Indicated | 24 hours) | 24 hours) | Point (hours) |
| Time Point added to | Mean ± Standard | Mean ± Standard | Mean ± Standard |
| Growth Media (10% TY) | Deviation (n = 3) | Deviation (n= 3) | Deviation (n = 3) |
| 10% TY | 0.15 ± 0.02 | 92 ± 10 | 9 ± 0 |
| 100/132 mM Arg/Pi | 0.25 ± 0.05 | 19 ± 5 | 23 ± 0 |
| 100/52 mM Arg/Pi | 0.34 ± 0.06 | 27 ± 8 | 19 ± 0 |
| 40/132 mM Arg/Pi | 0.34 ± 0.02 | 65 ± 11 | 23 ± 0 |
| 250/300 mM Arg/Pi | 0.135 ± 0.001 | 29.6 ± 0.4 | Did not reach |
| | | | inflection by 30.2 |
| | | | hours |
| 2% HPMC, 500/600 mM | 0.42 ± 0.07 | 5 ± 2 | 17 ± 0 |
| Arg/Pi - 22 h gel extract | | | |
| 2% HPMC, 500/600 mM | 0.43 ± 0.08 | 5 ± 3 | 17 ± 0 |
| Arg/Pi -48 h gel extract | | | |
| 2% HPMC, 500/262 mM | 0.36 ± 0.05 | 6 ± 3 | 13 ± 0 |
| Arg/Pi- 22 h gel extract | | | |
| 2% HPMC, 500/262 mM | 0.40 ± 0.07 | 6 ± 2 | 14 ± 0 |
| Arg/Pi- 48 h gel extract | | | |
| 2% HPMC, 200/660 mM | 0.54 ± 0.08 | 17 ± 3 | 15 ± 0 |
| Arg/Pi- 22 h gel extract | | | |
| 2% HPMC, 200/660 mM | 0.5 ± 0.1 | 19 ± 8 | 14 ± 0 |
| Arg/Pi- 48 h gel extract | | | |
| 2% HPMC- 48 h gel | 0.178 ± 0.008 | 30 ± 3 | 9 ± 0 |
| extract | | | |
| 0.5% HPMC, 500/660 | 0.07 ± 0.01 | 19 ± 7 | Did not reach |
| mM Arg/Pi gel | | | inflection by 30.2 |
| | | | hours |
| 0.5% HPMC, 200/262 | 0.18 ± 0.02 | 2 ± 3 | 23 ± 0 |
| Arg/Pi gel | | | |
| 0.5% HPMC gel | 0.20 ± 0.01 | 12 ± 2 | 10 ± 0 |
| 4% HPMC, 500/660 mM | 0.48 ± 0.09 | 8 ± 3 | 10 ± 0 |
| Arg/Pi- 22 h gel extract | | | |
| 4% HPMC, 500/660 mM | 0.57 ± 0.0.03 | 4 ± 2 | 10 ± 0 |
| Arg/Pi- 48 h gel extract | | | |
| 4% HPMC-22 h gel | 0.19 ± 0.03 | 79 ± 13 | 10 ± 0 |
| extract | | | |
| 4% HPMC-48 h gel | 0.23 ± 0.02 | 57 ± 7 | 10 ± 0 |
| extract | | | |

1. A method of suppressing microbial virulence, the method comprising administering and/or applying a medical composition, the medical composition comprising:
(a) between about 5 mM arginine and about 150 mM arginine; and
(b) between about 5 mM and about 150 mM of phosphate.

2. The method of claim 1, wherein the medical composition comprises an approximately equal molar ratio of arginine and phosphate.

3. The method of claim 1, wherein the medical composition comprises about 50 mM of arginine and about 50 mM phosphate.

4. The method of claim 1, wherein the medical composition comprises between about 25 mM of arginine and about 150 mM of arginine and between about 25 mM phosphate and about 150 mM phosphate.

5. The method of claim 1, wherein the medical composition is included in a spray, lotion, ointment, gel, solution, emulsion, dispersion, foam, coating, paste, powder, tablet, adhesive, or capsule.

6. The method of claim 1, wherein the medical composition further comprises a thickener.

7. The method of claim 6, wherein the thickener is selected from the group consisting of hydroxypropyl methyl cellulose, hydroxypropylcellulose, hydroxybutylmethylcellulose, hydroxyethylcelluloses, and any combinations thereof.

8. The method of claim 7, wherein the thickener comprises hydroxypropyl methylcellulose.

9. The method of claim 1, wherein administering and/or applying the medical composition suppresses at least one virulence factor.

10. The method of claim 9, wherein the virulence factor is pyoverdine or collagenase.

11. The method of claim 1, wherein a log reduction of microbes is less than 1.

12. The method of claim 1, wherein administrating and/or applying the medical composition prevents, mitigates, or treats a microbial infection.

13. The method of claim 1, wherein administrating and/or applying the medical composition comprises applying the medical composition to skin, mucosa, tissue, a wound site, a surgical site, an implant, catheter, suture, or a bone.

14. The method of claim 1 wherein administrating and/or applying the medical composition reduces or inhibits virulence of at least one of gram negative Pseudomonas aeruginosa, gram positive Enterococcus faecalis, gram positive Staphylococcus aureus, or gram negative Serratia marcescens.

15. A medical composition comprising:
(a) between about 5 mM arginine and about 150 mM arginine; and
(b) between about 5 mM and about 150 mM of phosphate.

16. The medical composition of claim 15, wherein the composition comprises an equal molar amount or more arginine than phosphate.

17. The medical composition of claim 15, wherein the composition comprises about 50 mM of arginine and about 50 mM phosphate.

18. The medical composition of claim 15, wherein the medical composition comprises between about 25 mM of arginine and about 150 mM of arginine and between about 25 mM phosphate and about 150 mM phosphate.

19. The medical composition of claim 15, wherein the composition is presented in a spray, lotion, ointment, gel, solution, emulsion, dispersion, foam, coating, paste, powder, tablet, adhesive, or capsule.

20. The method of claim 15, wherein the medical composition further comprises a thickener.

21.-34. (canceled)
